(19) United States
(12) Patent Application Publication
(10) Pub. No.: US 2016/0081659 A1
(43) Pub. Date: Mar. 24, 2016

(54) METHOD AND SYSTEM FOR SELECTING AN EXAMINATION WORKFLOW

(71) Applicant: General Electric Company, Schenectady, NY (US)

(72) Inventors: Christian Perrey, Mondsee (AT); Arthur Gritzky, Pollham (AT)

(21) Appl. No.: 14/495,422

(22) Filed: Sep. 24, 2014

(51) Int. Cl. A61B 8/00; A61B 8/08; A61B 19/00; A61B 8/06

(52) U.S. Cl. CPC A61B 8/463; A61B 8/466; A61B 8/06; A61B 8/469; A61B 8/4444; A61B 8/54; A61B 8/465; A61B 8/488; A61B 8/5223; A61B 19/56; A61B 2019/562

(57) ABSTRACT

A method and ultrasound imaging system for selecting an examination workflow for ultrasound imaging. The method and system include displaying a graphical model on a display device, selecting a modeled anatomical region in the graphical model that corresponds to an anatomical region in a patient. The method and system include automatically loading an examination workflow for the anatomical region, executing the examination workflow to acquire ultrasound data of the anatomical region, and generating and displaying a graphical output based on the ultrasound data on the display device.

### FIELD OF THE INVENTION

This disclosure relates generally to a method and system for selecting an examination workflow for ultrasound imaging with a graphical model.

### BACKGROUND OF THE INVENTION

For conventional ultrasound scanning, an operator needs to configure the ultrasound scanning parameters for each individual scan. This typically entails navigating through multiple menus in order to select acquisition presets such as imaging mode, line density, pulse repetition frequency, field-of-view, number of foci, frequency range, and display parameters for any resulting images. Many conventional systems require the user to access separate menus in order to individually select each preset or display parameter. Individually selecting each acquisition preset or display parameter can be a very time-consuming process for an operator of an ultrasound imaging system. Additionally, there is a risk of selecting a preset or display parameter that could significantly degrade the image quality of any resulting images or degrade the accuracy of any calculated values.

For these and other reasons an improved method and ultrasound imaging system for selecting an examination workflow are desired.

### BRIEF DESCRIPTION OF THE INVENTION

The above-mentioned shortcomings, disadvantages and problems are addressed herein which will be understood by reading and understanding the following specification.

In an embodiment, a method for selecting an examination workflow for ultrasound imaging with a graphical model includes displaying a graphical model on a display device where the graphical model represents at least a portion of a patient. The method includes selecting a modeled anatomical region in the graphical model, where the modeled anatomical region corresponds to an anatomical region in the patient. The method includes automatically loading an examination workflow for the anatomical region in response to selecting the modeled anatomical region and executing the examination workflow to acquire ultrasound data of the anatomical region. The method includes generating a graphical output based on the ultrasound data, and displaying the graphical output on the display device.

In an embodiment, an ultrasound imaging system includes a probe, a display device, and a processor in electronic communication with the probe and the display device. The processor is configured to display a graphical model on the display device, where the graphical model represents at least a portion of a patient. The processor is configured to receive a selection of a modeled anatomical region based on an input through the graphical model. The modeled anatomical region corresponds to an anatomical region. The processor is configured to automatically load an examination workflow for the anatomical region after receiving the selection of the modeled anatomical region. The processor is configured to control an acquisition of ultrasound data with the probe according to the examination workflow, generate a graphical output based on the ultrasound data, and display the graphical output on the display device.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment;

FIG. 2 is a flow chart of a method in accordance with an embodiment;

FIG. 3 is a schematic representation of a 3D graphical model in accordance with an embodiment;

FIG. 4 is a schematic representation of a 2D graphical model in accordance with an embodiment;

FIG. 5 is a schematic representation of a graphical model and a color flow image in accordance with an embodiment; and

FIG. 6 is a schematic representation of a graphical model and a b-mode image in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

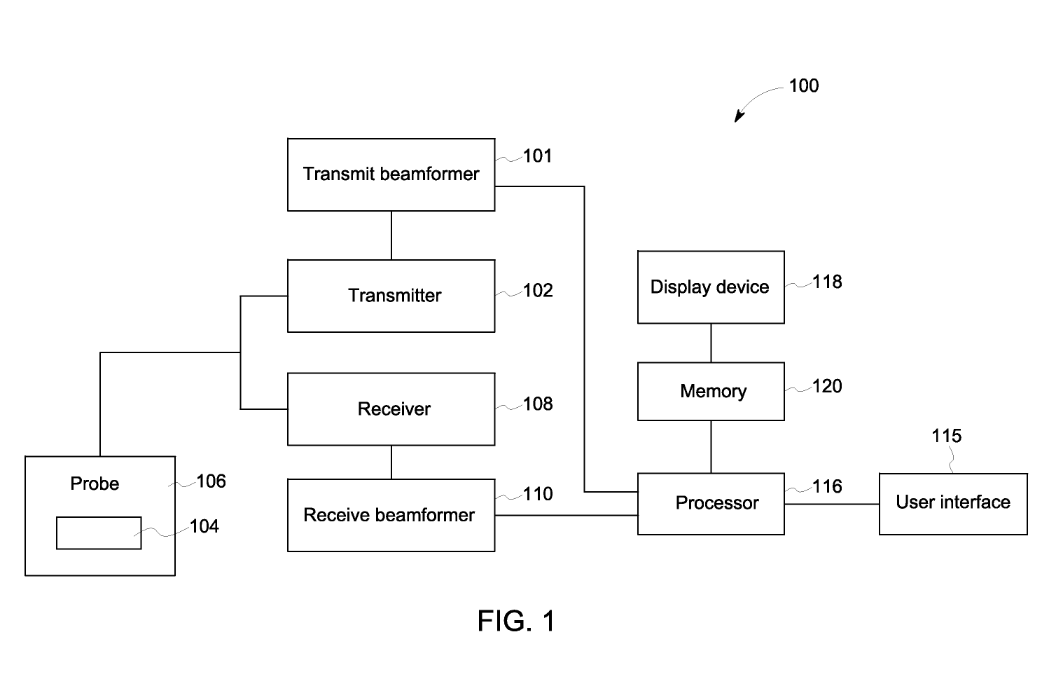

FIG. 1 is a schematic diagram of an ultrasound imaging system 100 in accordance with an embodiment. The ultrasound imaging system 100 includes a transmit beamformer 101 and a transmitter 102 that drive elements 104 within a probe 106 to emit pulsed ultrasonic signals into a body (not shown). The probe 106 may be a linear probe, a curved linear probe, a 2D array, a mechanical 3D/4D probe, or any other type of probe capable of acquiring ultrasound data. Still referring to FIG. 1, the pulsed ultrasonic signals are back-scattered from structures in the body, like blood cells or muscular tissue, to produce echoes that return to the elements 104. The echoes are converted into electrical signals by the elements 104 and the electrical signals are received by a receiver 108. The electrical signals representing the received echoes are passed through a receive beamformer 110 that outputs ultrasound data. According to some embodiments, the probe 106 may contain electronic circuitry to do all or part of the transmit and/or the receive beamforming. For example, all or part of the transmit beamformer 101, the transmitter 102, the receiver 108 and the receive beamformer 110 may be situated within the probe 106. The terms “scan” or “scanning” may also be used in this disclosure to refer to acquiring data through the process of transmitting and receiving ultrasonic signals. The terms “data” or “ultrasound data” may be used in this disclosure to refer to either one or more datasets acquired with an ultrasound imaging system. A user interface 115 may be used to control operation of the ultrasound imaging system 100, including, to control the input of patient data, to set an acquisition preset, or to change a display parameter, and the like. The user interface may include components such as a keyboard, a mouse, a track ball, a track pad, a touch screen, a multi-touch screen, and the like.

The ultrasound imaging system 100 also includes a processor 116 to control the transmit beamformer 101, the transmitter 102, the receiver 108 and the receive beamformer 110. The processor 116 is in electronic communication with the probe 106. The processor 116 may control the probe 106 to acquire data. The processor 116 controls which of the elements 104 are active and the shape of a beam emitted from the probe 106. The processor 116 is also in electronic communication with a display device 118, and the processor 116 may process the data into images or values for display on the display device 118. The display device 118 may comprise a monitor, an LED display, a cathode ray tube, a projector display, or any other type of apparatus configured for displaying an image. Additionally, the display device 118 may include one or more separate devices. For example, the display device 118 may include two or more monitors, LED displays, cathode ray tubes, projector displays, etc. For purposes of this disclosure, the term “electronic communication” may be defined to include both wired and wireless connections. The processor 116 may include a central processor (CPU) according to an embodiment. According to other embodiments, the processor 116 may include other electronic components capable of carrying out processing functions, such as a digital signal processor, a field-programmable gate array (FPGA), or a graphic board. According to other embodiments, the processor 116 may include multiple electronic components capable of carrying out processing functions. For example, the processor 116 may include two or more electronic components selected from a list of electronic components including: a central processor, a digital signal processor, an FPGA, and a graphic board. According to another embodiment, the processor 116 may also include a complex demodulator (not shown) that demodulates the RF data and generates raw data. In another embodiment the demodulation can be carried out earlier in the processing chain. The processor 116 may be adapted to perform one or more processing operations according to a plurality of selectable ultrasound modalities on the data. The data may be processed in real-time during a scanning session as the echo signals are received. For the purposes of this disclosure, the term “real-time” is defined to include a procedure that is performed without any intentional delay. For purposes of this disclosure, the term “real-time” will additionally be defined to include an action occurring within 2 seconds. For example, if data is acquired, a real-time display of that data would occur within 2 seconds of the acquisition. Those skilled in the art will appreciate that most real-time procedures/processes will be performed in substantially less time than 2 seconds. The data may be stored temporarily in a buffer (not shown) during a scanning session and processed in less than real-time in a live or off-line operation. The processor 116 may be able to load and execute a number of different workflows according to various embodiments. Each workflow may be configured for a specific type of ultrasound imaging exam. The workflows may, for instance, include a number of acquisition presets or display parameters for a particular type of ultrasound exam. Acquisition presets may, for example, include parameters such as ultrasound imaging mode, line density, pulse-repetition frequency (PRF), field of view, number of foci, position of focus or foci, frequency range, and the like. Display parameters may include display parameters such as window width, window level, gain, display format, and the like.

Some embodiments of the invention may include multiple processors (not shown) to handle the processing tasks. For example, a first processor may be utilized to demodulate and decimate the RF signal while a second processor may be used to further process the data prior to displaying an image. It should be appreciated that other embodiments may use a different arrangement of processors.

The ultrasound imaging system 100 may continuously acquire data at a given frame-rate or volume-rate. Images generated from the data may be refreshed at a similar frame-rate or volume-rate. A memory 120 is included for storing processed frames of acquired data. In an exemplary embodiment, the memory 120 is of sufficient capacity to store at least several seconds' worth of frames of ultrasound data. The frames of data are stored in a manner to facilitate retrieval thereof according to its order or time of acquisition. The memory 120 may comprise any known data storage medium.

Optionally, embodiments of the present invention may be implemented utilizing contrast agents. Contrast imaging generates enhanced images of anatomical structures and blood flow in a body when using ultrasound contrast agents including microbubbles. After acquiring data while using a contrast agent, the image analysis includes separating harmonic and linear components, enhancing the harmonic component and generating an ultrasound image by utilizing the enhanced harmonic component. Separation of harmonic components from the received signals is performed using suitable filters. The use of contrast agents for ultrasound imaging is well-known by those skilled in the art and will therefore not be described in further detail.

In various embodiments of the present invention, data may be processed by other or different mode-related modules by the processor 116 (e.g., B-mode, Color Doppler, M-mode, Color M-mode, spectral Doppler, Elastography, TVI, strain, strain rate, and the like) to form 2D or 3D data. For example, one or more modules may generate B-mode, color Doppler, M-mode, color M-mode, spectral Doppler, Elastography, TVI, strain, strain rate and combinations thereof, and the like. The image beams and/or frames are stored and timing information indicating a time at which the data was acquired in memory may be recorded. The modules may include, for example, a scan conversion module to perform scan conversion operations to convert the image frames from coordinates beam space to display space coordinates. A video processor module may be provided that reads the image frames from a memory and displays the image frames in real time while a procedure is being carried out on a patient. A video processor module may store the image frames in an image memory, from which the images are read and displayed.

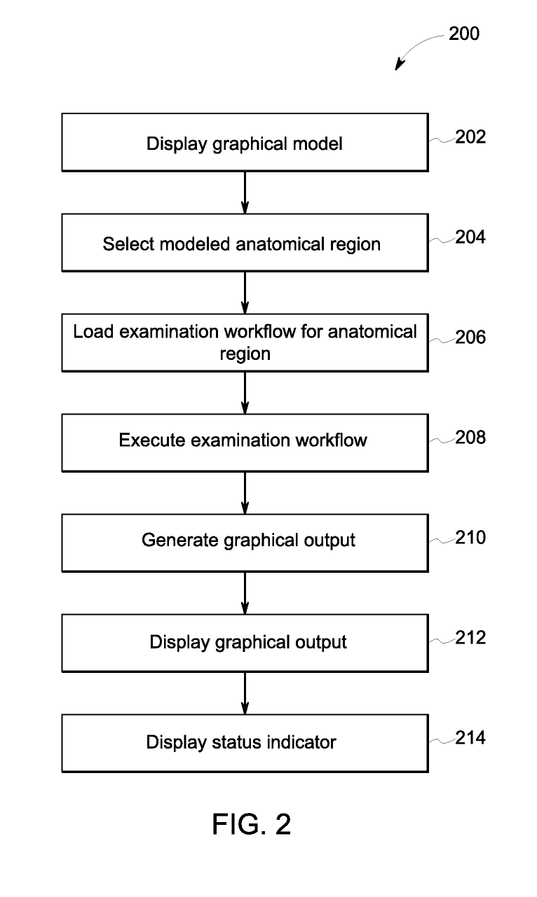

FIG. 2 is a flow chart of a method 200 in accordance with an exemplary embodiment. The individual blocks of the flow chart represent steps that may be performed in accordance with the method 200. Additional embodiments may perform the steps shown in a different sequence and/or additional embodiments may include additional steps not shown in FIG. 2. The technical effect of the method 200 is the execution of an examination workflow and the display of a graphical output based on the selection of a modeled anatomical region in a graphical model. The method 200 will be described in detail hereinafter.

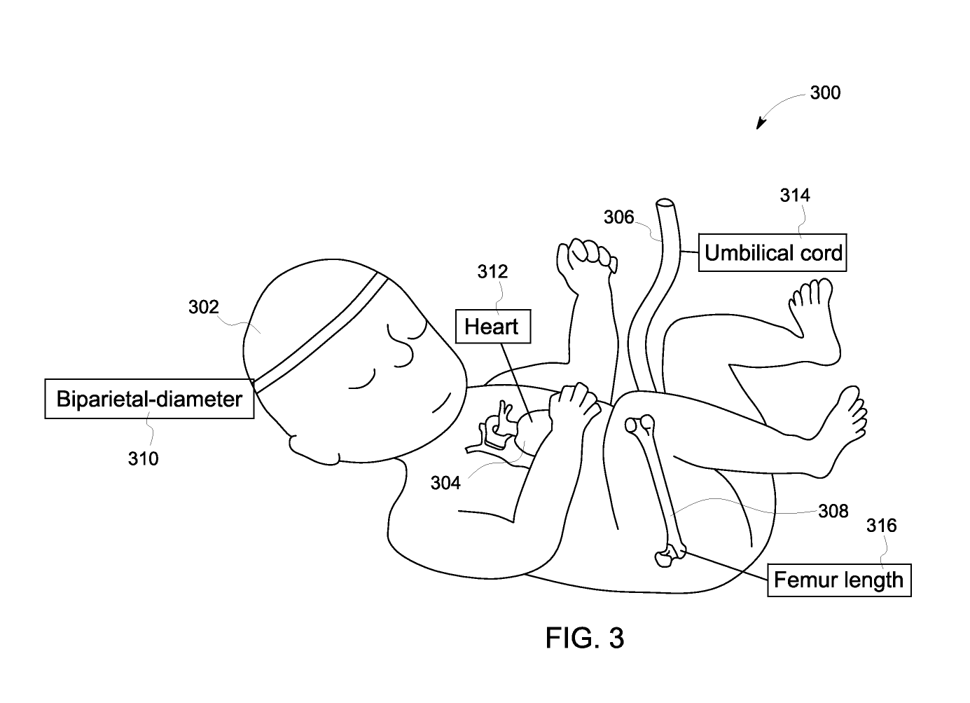

FIG. 3 is a schematic representation of a 3D graphical model 300 according to an embodiment. The 3D graphical model 300 is a representation of a fetus and it is adapted to be used as a component of a graphical user interface. The 3D graphical model 300 includes a plurality of modeled anatomical regions, each of which may be selected in order to select an examination workflow. Both the modeled anatomical regions and the examination workflow will be described in additional detail hereinafter.

FIG. 2 will be described according to an exemplary embodiment where the method 200 is performed using the ultrasound imaging system 100 shown in FIG. 1 and the 3D graphical model 300 shown in FIG. 3. At step 202, the processor 116 displays a graphical model, such as the 3D graphical model 300, on the display device 118. The 3D graphical model 300 represents an entire fetus, but other graphical models may represent just a portion of a patient. Additionally, a graphical model may represent a child or an adult patient in accordance with various embodiments. The graphical model may represent an average patient. For example, the graphical model may be based on a statistically average patient, a representative patient in a particular demographic, or the graphical model may be a schematic graphical representation of at least a portion of a patient. The graphical model may be very life-like in appearance or the graphical model may be a less-accurate representation of at least a portion of a patient. The 3D graphical model 300 may optionally be rotated in 3 dimensions, including a rotation direction that is not in the plane of the display surface, to change a viewing angle of the 3D graphical model 300 in order to facilitate the selection of various modeled anatomical regions represented on the 3D graphical model 300. A 2D graphical model may be displayed according to other embodiments. An example of a 2D graphical model will be described hereinafter.

At step 204, a user selects a modeled anatomical region from the anatomical model such as the 3D anatomical model 300. The 3D anatomical model 300 includes four modeled anatomical regions that are labeled in FIG. 3. The 3D anatomical model 300 includes a modeled head region 302, a modeled heart region 304, a modeled umbilical cord region 306, and a modeled femur region 308. FIG. 3 also includes labels indicating the examination workflow associated with the modeled anatomical region. For example, the modeled head region 302 includes a first label 310 indicating that the examination workflow is for biparietal diameter. The modeled heart region 304 includes a second label 312 indicating that the examination workflow is for the heart. The modeled umbilical cord region 306 includes a third label 314 indicating that the examination workflow is for the umbilical cord. The modeled femur region 308 includes a fourth label 316 indicating that the examination workflow is for femur length.

The user may select one of the modeled anatomical regions represented in the 3D graphical model 300. According to an exemplary embodiment, the user may select the modeled anatomical region by positioning a cursor or a pointer over the desired modeled anatomical region and clicking the modeled anatomical region to select it. According to an embodiment where the 3D graphical model is displayed on a screen that functions as a touch screen or a multi-touch screen, the user may select the desired modeled anatomical region by taping on the portion of the desired modeled anatomical region on the screen. While the 3D graphical model 300 includes a first label 310, a second label 312, a third label 314, and a fourth label 316, it should be appreciated that all of the labels may not always be shown at the same time as the 3D graphical model 300. For example, the labels may only be displayed when the user positions a cursor or pointer over the modeled anatomical region associated with the particular label. For example, the first label 310 may only be displayed when the user “hovers” the cursor or pointer over the modeled head region 302. The labels may also only be shown when the user clicks or taps a single time on the modeled anatomical region. The user may need to click or tap on the modeled anatomical region a second time or with a double-click/double-tap to select the examination workflow. Only showing one label at a time results in a less cluttered graphical model. A modeled anatomical region may be selected from the graphical model in other ways according to various embodiments.

Next, at step 206, the processor loads the examination workflow for an anatomical region corresponding to the selected modeled anatomical region. For purposes of this disclosure, an anatomical region is said to correspond to a modeled anatomical region if the anatomical region is of the same anatomical structure represented in the modeled anatomical region. Referring to FIG. 3, if the user were to select the modeled head region 302, the processor 116 would load a workflow for biparietal diameter of the fetus's head. The biparatel diameter is a diameter measurement of the fetus's head used to chart growth. If the user were to select the modeled heart region 304, the processor would load a workflow for a heart or cardiac scan. For example, the cardiac workflow may include the settings for a volume acquisition using spatio-temporal image correlation (STIC) and subsequent display of any acquired images. If the user were to select the modeled umbilical cord region 306, the processor 116 would load an examination workflow for analyzing flow of the umbilical cord. For example, the processor 116 may load an examination workflow for acquiring and displaying a color flow image. If the user were to select the modeled femur region 308, the processor 116 would load the workflow to acquire an image used for calculating femur length.

The step of loading the examination workflow may mean many different things according to various embodiments. The examination workflow may include a series of steps needed to perform a particular examination. Loading the examination workflow may include a setting a plurality of acquisition presets and/or display parameters for the specific examination. Acquisition presets may include parameters such as ultrasound imaging mode, line density, pulse-repetition frequency (PRF), field of view, number of foci, position of focus or foci, frequency range, etc. Display parameters may include display parameters such as window width, window level, gain, and display format. The processor 116 may automatically configure the acquisition presets and display parameters for acquiring and displaying ultrasound data for the examination associated with the selected modeled anatomical region. By interfacing with the graphical model, the user is presented with a very intuitive way to select and load a desired examination workflow. With very few clicks or inputs, the user is able to select and load the examination workflow for a desired type of scan or examination. This saves time for the operator. Additionally, the graphical model provides a consistent way to achieve appropriate acquisition presets and display parameters for a particular type of examination. Additionally, the graphical model makes selecting the desired examination workflow easier and faster for new or less-skilled users since they can leverage the various modeled anatomical regions represented in the graphical model to help guide the selection of the most appropriate examination workflow for the desired anatomical region. Also, by simply selecting the modeled anatomical region with a very small number of inputs, the user can load the entire examination workflow.

At step 208, the examination workflow is executed. The examination workflow may be executed by the processor 116, by the operator, or by a combination of the processor 116 and the operator. For example, according to an embodiment, executing the examination workflow may include acquiring ultrasound data using the acquisition presets that were loaded during step 206. For multi-step examination workflows, executing the examination workflow may include performing all of the steps, whether the steps require acquiring ultrasound data or manually interacting with acquired ultrasound data, in order to complete the multi-step examination workflow.

Next, at step 210, the processor generates a graphical output. The graphical output may be the result of the examination workflow, such as an image, a measurement, or a value generated from ultrasound data. At step 212, the processor 116 displays the graphical output on the display device 118. Examples of various graphical outputs will be provided hereinafter. According to an embodiment, at step 214 the processor 116 may display a status indicator on the graphical model, such as the 3D graphical model 300 to indicate a completion status for the examination workflow. For example, if an examination workflow has been completed, the processor may display a status indicator to represent that a particular examination workflow has been completed. The status indicator may include a colorization. For example, the modeled anatomical region associated with an examination workflow may be colorized a color such as green to indicate that the examination workflow has been completed. The status indicator may also optionally indicate if a particular examination workflow has not been completed. According to an embodiment, the status indicator may also indicate if a particular examination workflow is “in progress.” For example, an examination workflow that is “in progress” may be colorized with a different color, such as yellow. Examination workflows that are not “in progress” and that are not completed may be colorized with a different color or they may simply not be colorized. According to other embodiments, the status indicator may include displayed text indicating that the examination workflow has been completed. Or, the status indicator may include an icon to symbolize that a particular examination workflow has been completed. The status indicator may be displayed on or near a particular modeled anatomical region on the graphical model to clearly indicate which examination workflow has been completed.

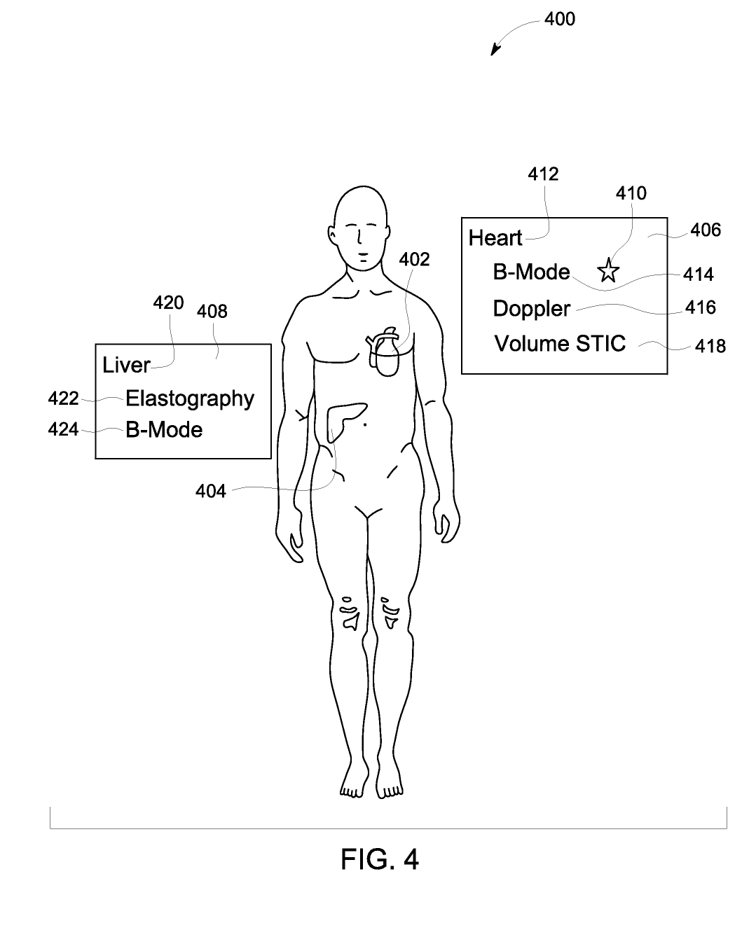

FIG. 4 is a schematic representation of a 2D graphical model 400 in accordance with an embodiment. Unlike the 3D graphical model 300 described previously, the 2D graphical model may only be rotated within the plane of the display device. Two different modeled anatomical regions are schematically represented on the 2D graphical model: a modeled heart region 402 and a modeled liver region 404. It should be appreciated that other modeled anatomical regions may be represented on 2D graphical models according to other embodiments.

FIG. 4 includes a first menu 406, a second menu 408, and an icon 410. According to an embodiment, the first menu 406 may only be displayed when a user selects the modeled heart region 402 and the second menu 408 may only be displayed when a user selects the modeled liver region 404. The user may, for instance, select the anatomical region by clicking on the desired modeled anatomical region or by hovering a cursor or a pointer over the desired modeled anatomical region. The first menu 406 includes a title 412, a first examination workflow 414, a second examination workflow 416, and a third examination workflow 418. The title 406 indicates that the modeled anatomical region is the heart; the first examination workflow 414 is for a B-mode examination; the second examination workflow 416 is for a Doppler examination; and the third examination workflow 418 is for a Volume STIC acquisition. The second menu 408 includes a title 420, a first examination workflow 422, and a second examination workflow 424. According to an exemplary embodiment, the user may first select a modeled anatomical region, such as by selecting the modeled heart region 402, and then the first menu 406 may be displayed. The user may next select the desired examination workflow from the possible examination workflows displayed in the first menu 406. The possible examination workflows, that is, the first examination workflow 414, the second examination workflow 416, and the third examination workflow 418, may represent various examination workflows that may be executed with respect to the heart. Likewise, the first examination workflow 422 and the second examination workflow 424 may be executed with respect to the liver examination region. The various examination workflows may be displayed in a drop-down menu, as shown in FIG. 4, or in other configurations according to other embodiments. The embodiment shown in FIG. 4 provides an efficient and intuitive way for the user to select the desired examination workflow for a selected anatomical region. FIG. 4 also includes an icon 410. The icon 410 is a star according to an embodiment. The icon 410 is displayed with the first examination workflow 414 (i.e. the B-mode examination) to indicate that the B-mode examination workflow has been completed. The icon 410 is just one example of a status indicator that may be displayed to indicate the completion status of various examination workflows. While the icon 410 is a star according to the embodiment of FIG. 4, it should be appreciated that any other icon may be used to indicate the completion status of a particular workflow according to other embodiments.

The embodiment of FIG. 4 shows various examination workflows that may be associated with each anatomical region. However, in other embodiments menus may include a plurality of steps that need to be performed in order to complete a multi-step examination workflow. A status indicator, such as an icon or a colorization may be used to graphically indicate the completion status of each step in the multi-step workflow. It should be appreciated that status indicators may be displayed with 3D graphical models, such as the 3D graphical model 300 shown in FIG. 3.

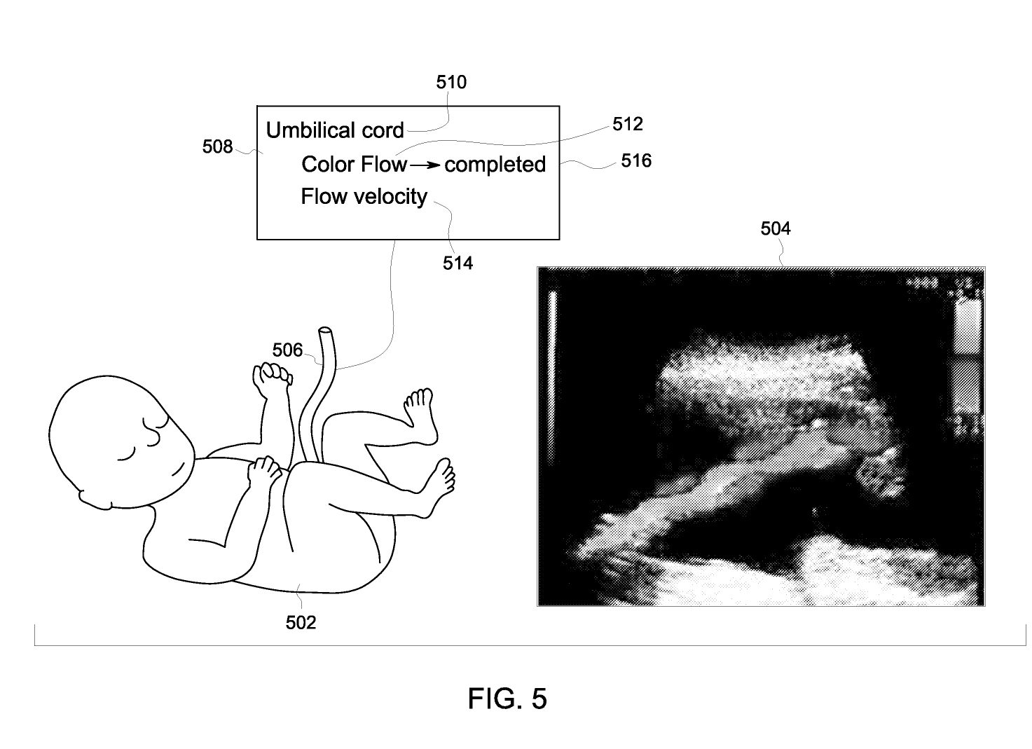

FIG. 5 is a schematic representation of a 3D graphical model 502 and a color flow image 504 in accordance with an exemplary embodiment. The 3D graphical model 502 represents a fetus and includes a modeled anatomical region 506 that is an umbilical cord. A menu 508 includes a title 510, a first examination workflow 512, and a second examination workflow 514. A text-based message 516 is used as a status identifier to indicate that the first examination workflow (i.e. the color flow examination workflow) has been completed. The color flow image 504 represents the graphical output that was generated and displayed by executing the first examination workflow 512. According to another embodiment, the first examination workflow 512 and the second examination workflow 514 may represent two steps of a multi-step examination workflow.

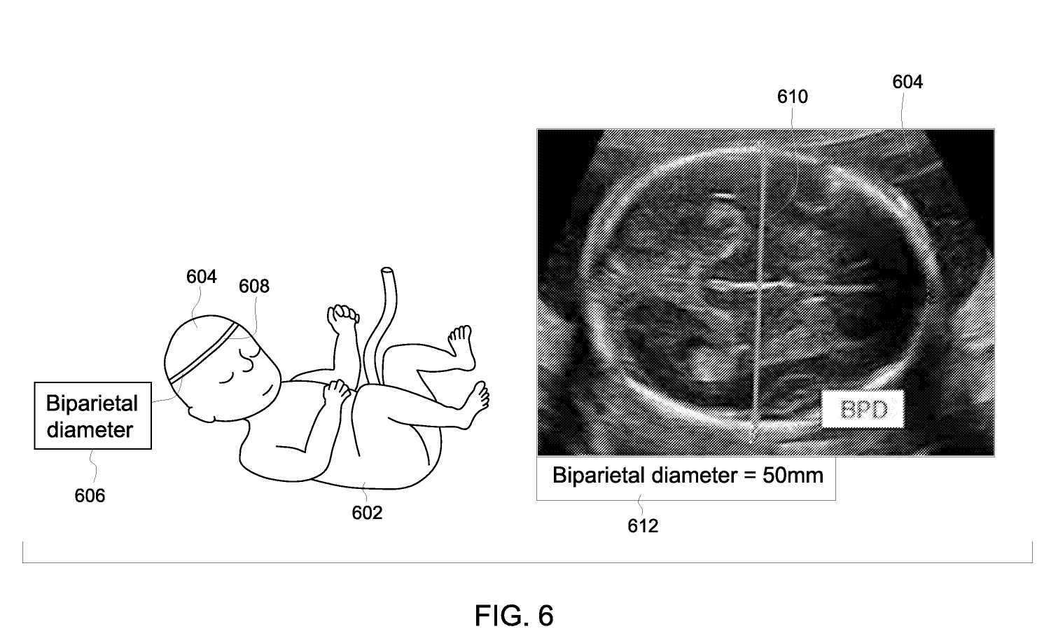

FIG. 6 is a schematic representation of a 3D graphical model 602 and b-mode image 604 in accordance with an exemplary embodiment. The 3D graphical model 602 represents a fetus and includes a modeled anatomical region 604. The modeled anatomical region 604 may be the head of a fetus. The 3D graphical model 602 also includes a graphical representation 608. Both the graphical representation 608 and the menu 606 may indicate that the selected examination workflow is for calculating the biparietal diameter. The b-mode image 604 is a b-mode image acquired according to an examination workflow used to calculate the biparietal diameter. The b-mode image 604 includes a symbol 610 representing the biparietal diameter and a measurement value 612. The measurement value 612 is 50 mm according to an embodiment.

According to an embodiment, the graphical output generated by following the examination workflow 606 may include one or both of the b-mode image 604 and the measurement 612. It should be appreciated that the measurement 612 representing the biparietal diameter is just one example of a measurement. Measurements or values relating to many different processes and/or functions may be displayed as the graphical output according to other embodiments. A non-limiting list of various measurements and values includes: lengths, such as femur length; distances, such as biparietal diameter; volumes, such as end-diastole volume or end-systole volume for cardiac applications; flow rate, such as for cardiac or vascular applications; flow volume; and tissue stiffness.

In addition to the examination workflows described above, some examination workflows may involve multiple separate acquisitions and processing steps that need to be performed to complete the examination. For workflows such as these, which will hereinafter be referred to as multi-step examination workflows, the processor 116 may automatically load the acquisition presets and display parameters for each subsequent examination after the previous examination has been completed. This way, the user can easily progress through all the individual steps of the multi-step examination workflow without having to manually adjust any of the acquisition presets or display parameters. Additionally, the examination workflow may guide the user through the actions needed to complete all the steps in the multi-step examination workflow. For example, the processor my display prompts on the display device 118 in order to alert the user of upcoming steps needed to complete all the steps of the multi-step examination workflow.

According to another embodiment, selecting a modeled anatomical region may provide the user with multiple options regarding examination workflows that may be selected or initiated. For example, a plurality of measurements, images, or a combination of measurements and images may all be associated with the same group even though some of the measurements and/or images may be associated with different anatomical regions. For example, when determining the growth progression of a fetus, a number of growth measurements may be used. The growth measurements may include biparietal diameter, head circumference, abdominal circumference, femur length, and humerus length. Biparietal diameter and head circumference are both associated with the modeled anatomical region of the head. Abdominal circumference is associated with the modeled anatomical region of the abdomen. Femur length is associated with the modeled anatomical region of the femur, and humerus length is associated with the modeled anatomical region of the humerus. Or, according to some embodiments, both the femur length and the humerus length may be more generally associated with the modeled anatomical region of the leg.

The user may select the modeled anatomical region by techniques such as clicking or tapping on the desired anatomical region. Or, the user may hover a cursor or pointer over the desired anatomical region in order to display a drop-down menu showing various options for examination workflows associated with the selected anatomical regions. After selecting one of the modeled anatomical regions in a group, such as any of the aforementioned modeled anatomical regions associated with growth measurements, the user may have the option to perform an examination workflow just for the selected anatomical region. Or, the user may have the option to select an multi-step examination workflow that initiates examination workflows for multiple anatomical regions. For example, after selecting the modeled anatomical region of the femur, the user may have the option to select an examination workflow for the femur length, or the user may have the option to select an examination workflow for all of the growth measurements since femur length belongs to the “growth measurements” group. Selecting the growth measurements examination workflow would start a workflow to acquire, for example, biparietal diameter, head circumference, abdominal circumference, and humerus length in addition to femur length. It should be appreciated that this is just one exemplary embodiment of a group and that examination workflows may be organized into groups in different manners according to other embodiments.

According to another embodiment, the examination workflows may be arranged in a hierarchical manner on the graphical model. For example, selecting a first modeled anatomical region may result in the display of a more-detailed view of the selected modeled anatomical region. The more-detailed view of the modeled anatomical region may be, for instance, a zoomed-in view of the modeled anatomical region. The more-detailed view of the modeled anatomical region may include a more-detailed view of the anatomy in the selected modeled anatomical region. The user may then select a modeled anatomical region and an associated examination workflow from the more-detailed view. The graphical model in the embodiment described above has two different levels: a normal view and a more-detailed view. However, it should be appreciated that other embodiments may include graphical models with more than two different levels. For example, it may be possible to iteratively select a modeled anatomical region, view a more-detailed view of the modeled anatomical region, and then select an new modeled anatomical region from the more-detailed view multiple times if high resolution or magnification is needed to select the desired modeled anatomical region. Multiple levels may also be used if there are too many examination workflow associated with a single modeled anatomical structure to be easily displayed at the same time.

For example, the user may hover or click on the head in a graphical model. In response to selecting the head, a more-detailed graphical model of the head may be displayed. The more-detailed graphical model may include labels for examination workflows such as biparietal diameter, head circumference, and the brain. According to an exemplary embodiment, the user may select the brain and then a more-detailed graphical model of the brain may be displayed including labels such as cerebellar diameter and cisterna magna depth. It should be appreciated that this is just one example of how a user may interact with a graphical model. Graphical models may include different anatomical regions, different levels, and different examination workflows according to other embodiments.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method for selecting an examination workflow for ultrasound imaging with
a graphical model, the method comprising:
displaying a graphical model on a display device, the graphical model representing at least a portion of a patient;
selecting a modeled anatomical region in the graphical model, the modeled anatomical region corresponding to an anatomical region in the patient;
automatically loading an examination workflow for the anatomical region in response to selecting the modeled anatomical region;
executing the examination workflow to acquire ultrasound data of the anatomical region;
generating a graphical output based on the ultrasound data; and
displaying the graphical output on the display device.

2. The method of claim 1, wherein the graphical model comprises a 2D graphical model.

3. The method of claim 1, wherein the graphical model comprises a 3D graphical model.

4. The method of claim 1, wherein the graphical output comprises an ultrasound image.

5. The method of claim 1, wherein the graphical output comprises a measurement value or a flow value.

6. The method of claim 1, wherein selecting the modeled anatomical region comprises clicking or tapping on the modeled anatomical region in the graphical model.

7. The method of claim 1, wherein selecting the modeled anatomical region comprises clicking or tapping on a first portion of the graphical model, displaying a more-detailed view including the modeled anatomical region, and then selecting the modeled anatomical region from the more-detailed view.

8. The method of claim 1, wherein said automatically loading the examination workflow comprises automatically setting a plurality of acquisition presets for acquiring the ultrasound data of the anatomical region.

9. The method of claim 8, wherein the plurality of acquisition presets are selected from a list of acquisition presets including: an ultrasound imaging mode, a line density, an image quality, a field-of-view, a number of foci, and a frequency range.

10. The method of claim 1, wherein said automatically loading the examination workflow comprises setting a display parameter.

11. The method of claim 1, further comprising displaying a status indicator on the graphical model to indicate a completion status of the examination workflow.

12. The method of claim 11, wherein the examination workflow comprises a plurality of steps, and wherein the status indicator further indicates a completion status for each of the plurality of steps of the examination workflow.

13. An ultrasound imaging system comprising:
a probe;
a display device; and
a processor in electronic communication with the probe and the display device, wherein the processor is configured to:
display a graphical model on the display device, the graphical model representing at least a portion of a patient;
receive a selection of a modeled anatomical region based on an input through the graphical model, the modeled anatomical region corresponding to an anatomical region;
automatically load an examination workflow for the anatomical region after receiving the selection of the modeled anatomical region;
control an acquisition of ultrasound data with the probe according to the examination workflow;
generate a graphical output based on the ultrasound data; and
display the graphical output on the display device.

14. The ultrasound imaging system of claim 13, wherein the examination workflow comprises a plurality of acquisition presets.

15. The ultrasound imaging system of claim 13, wherein the examination workflow comprises a display parameter used with the display device.

16. The ultrasound imaging system of claim 13, wherein the processor is further configured to display a status indicator with the graphical model to indicate a completion status of the examination workflow.

17. The ultrasound imaging system of claim 13, wherein the processor is further configured to display a plurality of status indicators on the graphical model, each status indicator indicating a completion status of a different examination workflow.

18. The ultrasound imaging system of claim 16, wherein the processor is further configured to display a status indicator on the graphical model to indicate completion status for each of a plurality of steps of the examination workflow.

19. The ultrasound imaging system of claim 16, wherein the status indicator comprises a color.

20. The ultrasound imaging system of claim 16, wherein the status indicator comprises an icon.
